(19) United States
(12) Patent Application Publication
(10) Pub. No.: US 2005/0232992 A1
(43) Pub. Date: Oct. 20, 2005

(54) Proton pump inhibitor formulations, and methods of preparing and using such formulations

(75) Inventors: John Devane, Roscommon (IE); Jackie Butler, Westmeath (IE); Paul Stark, Westmeath (IE)

Correspondence Address: GREENBLUM & BERNSTEIN, P.L.C., 1950 ROLAND CLARKE PLACE, RESTON, VA, 20191, US

(73) Assignee: AGI Therapeutics Limited, Roscommon (IE)

(21) Appl. No.: 10/932,098

(22) Filed: Sep. 2, 2004

Related U.S. Application Data
(60) Provisional application No. 60/499,362, filed on Sep. 3, 2003.

(51) Int. Cl. A61K 9/54; A61K 9/22

(52) U.S. Cl. 424/468

(57) ABSTRACT

Pharmaceutical formulation comprising at least one proton pump inhibitor structured and arranged to provide an initial pH independent time-based delayed-release, and a subsequent extended-release of the at least one proton pump inhibitor.

### CROSS REFERENCE TO RELATED APPLICATIONS

This application claims the benefit of U.S. Provisional Patent Application No. 60/499,362, in the names of John Devane et. al., entitled “Proton Pump Inhibitor Formulations, and Methods of Preparing and Using Such Formulations”, filed on Sep. 3, 2003 the disclosure of which is expressly incorporated herein by reference as though set forth in full herein.

### BACKGROUND OF THE INVENTION

1. Field of the Invention

The present invention is directed to proton pump inhibitors (PPI's), to formulations containing proton pump inhibitors, to formulations containing proton pump inhibitors that are constructed and arranged to provide unique dissolution profiles, and particularly to formulations designed to treat gastric acid related conditions, especially to counteract nocturnal acid breakthrough. The formulations according to the present invention particularly comprise proton pump inhibitor formulations that have a pH independent time-delayed release followed by a sustained release. The present invention is also directed to methods of using proton pump inhibitors, such as in the treatment of gastric acid related conditions, including methods wherein the proton pump inhibitor is administered in a formulation that provides for pH independent time delay of the release of the proton pump inhibitor followed by sustained release of the proton pump inhibitor. The formulations of the present invention can be used to treat nocturnal acid breakthrough, either alone or in combination with other formulations. The present invention is also directed to methods of preparing such formulations.

2. Discussion of Background Information

Proton pump inhibitors are commonly used to treat gastric acid related conditions such as ulcers, gastritis, GERD (Gastroesophageal Reflux Disease), including erosive and non-erosive reflux desease, dyspepsia, with or without heartburn, and Barretts esophogeus. Proton pump inhibitors (PPI) are commonly used as monotherapy, either as once-daily or twice daily dosing. Proton pump inhibitors are also used in combination with H₂RA (histamine₂ receptor antagonists) and antibiotics, particularly in helicobacter positive patients.

Despite their success, proton pump inhibitors have not been wholly effective in treating all patients and there is, in particular, a significant number of patients on proton pump inhibitors (up to about 73%) who experience nocturnal acid breakthrough (NAB). Nocturnal acid breakthrough is defined as a gastric pH of less than 4 for any 1 hour period between 10:00 PM and 8:00 AM, which is contrasted with daytime acid breakthrough (DAB) which is defined as a gastric pH of less than 4 for any 1 hour period between 8:00 AM and 10:00 PM. See, for example, Nzeako et al., Aliment. Pharmacol. Ther., “An Evaluation of the Clinical Implications of Acid Breakthrough in Patients on Proton Pump Inhibitor Therapy”, 2002: 16,1309-1316, the disclosure of which is incorporated by reference herein in its entirety.

The pattern of NAB is consistent with a “circadian” pattern, i.e., NAB occurs typically at about 1:00 AM, and extends until approximately 5:00 AM (Katz et al., Curr. Gastroenterol., “The Pharmacology and Clinical Relevance of Proton Pump Inhibitors”, 2002:4,459-462 and Peghini et al., Gastroenterology, “Ranitidine Controls Nocturnal Gastric Acid Breakthrough on Omeprazole: A Controlled Study in Normal Subjects”, 1998:115, 1335-1339, the disclosures of which are incorporated by reference herein in their entireties. While the exact basis of this pattern is unclear it has been proposed that it reflects a circadian rhythm in terms of synthesis and processing of the proton pump with the appearance of new pumps at night (Hirschowitz et al., Digestive Diseases and Sciences, Vol. 40, No. 2 (February 1995 Supplement), pp.3S-23S, the disclosure of which is incorporated by reference herein in its entirety).

Efforts at modifying proton pump inhibitor dosing to control NAB are disclosed in the literature to have only had limited success. Thus, dosing of a proton pump inhibitor at night (either at dinner time—typically 6:00 PM) or at bed-time (typically 10:30 PM) have not eliminated or significantly reduced the incidence of NAB (Ours et al., The American Journal of Gastroenterology, “Nocturnal Acid Breakthrough: Clinical Significance and Correlation With Esophageal Acid Exposure”, Vol. 98, No. 3, 2003, pp. 545-550, and Nzeako et al., Aliment. Pharmacol. Ther., “An Evaluation of the Clinical Implications of Acid Breakthrough in Patients on Proton Pump Inhibitor Therapy”, 2002: 16,1309-1316, the disclosures of which are incorporated by reference herein in their entireties). Without wishing to be bound by theory, the reason for this is believed to be related to the short half-life of the proton pump inhibitor (for example, omeprazole typically has a half-life of approximately 50 minutes), and the need for the proton pump inhibitor to penetrate into the actively secreting parietal cell and acid dependently concentrate in the canalicuius, convert to its active form and bind the proton pump (Hirschowitz et al., Digestive Diseases and Sciences, Vol. 40, No. 2 (February 1995 Supplement), pp.3S-23S, the disclosure of which is incorporated by reference herein in its entirety). Thus, the systemic availability of the proton pump inhibitor after evening or night-time dosing is not aligned to the “circadian” pattern of acid secretion and will be largely metabolized and eliminated prior to the NAB peak.

One suggested manner of treating NAB, such as disclosed by Xue et al., Aliment Pharmacol. Ther. 2001:15:1351-1356, the disclosure of which is incorporated by reference herein in its entirety, is by adding an H₂RA at bedtime to the proton pump inhibitor. It is suggested by Xue et al. that this dual approach can enhance nocturnal gastric acid pH control, decrease nocturnal gastric acid breakthrough, and decrease the duration of oesphageal acid reflux associated with NAB.

As can be seen from the above, pharmaceutical formulation efforts to date have not been directed to achieving a release profile of proton pump inhibitors that align with the NAB pattern. Indeed, pharmaceutical formulations to date have focused on the specific gastric acid labile characteristics of the proton pump inhibitors. Thus, formulations have been described that “protect” the proton pump inhibitor within an enteric coating layer. It appears that many formulations in the prior art consider it necessary to include in proton pump inhibitor dosage forms an enteric coating. In other words, it appears that many current proton pump inhibitor dosage forms include, as a critical element, an enteric polymer system that is triggered to dissolve and release the proton pump inhibitor under intestinal pH conditions.

In particular, many formulations in the prior art include pH control of the release of the proton pump inhibitor in order that the proton pump inhibitor is released under more neutral or alkaline conditions. See, for example, U.S. Pat. No. 4,786,505 to Lovgren et al., U.S. Pat. No. 4,853,230 to Lovgren et al., U.S. Pat. No. 5,690,960 to Bengtsson et al., U.S. Pat. No. 5,817,338 to Bergstrand et al., U.S. Pat. No. 6,207,198 to Seth, U.S. Pat. No. 6,248,810 to St. Clair et al., U.S. Pat. No. 6,248,355 to Seth, the disclosures of which are incorporated by reference herein in their entireties.

The use of enteric polymers has then also led to the associated need to separate the proton drug inhibitor from the acid moieties of the enteric polymer by the insertion of an intermediate non-enteric “barrier” coating. This barrier layer is critical for shelf-life stability of the enteric based formulations. Most commonly this barrier coat is a water-soluble layer and these formulations are designed to rapidly release the proton pump inhibitor on emptying from the stomach.

In U.S. Pat. Nos. 5,945,124, 6,068,856 and 6,274,173 to Sachs et al., the disclosures of which are incorporated by reference herein in their entireties, , there is disclosed at least one release-slowing, release-controlling intermediate layer. In particular, Sachs et al. is directed to enhancing of the action of an antimicrobially-active ingredient on Helicobacter by administering pantoprazole in slow-release dosage form (extended release form). Sachs et al. disclose that it is surprising that administration of slow-release pantoprazole results in onset of action taking place significantly faster than on administration in a form without retarding such release, and that the duration of treatment until Helicobacter is eradicated is shortened, saving considerable amounts of antibiotic and acid inhibitor.

Sachs et al. disclose that their invention relates to oral pharmaceutical compositions in pellet or tablet form for combined use of pantoprazole with an antimicrobially-active ingredient for treatment of disorders caused by Helicobacter, wherein pantoprazole is present at least partly in slow-release form. Moreover, Sachs et al. disclose that their invention also relates to an oral pharmaceutical composition in pellet or tablet form for acid-labile irreversible proton pump inhibitors comprising an alkaline pellet or tablet core, at least one release-slowing, release-controlling intermediate layer and an outer enteric layer which is soluble in the small intestine, wherein the intermediate layer for the pharmaceutical composition is formed from a water-insoluble film former, the film former being applied from anhydrous solution or aqueous dispersion.

The goal of the system of Sachs et al. is not directed to treating NAB and is not directed to any time-course and release profile to align proton pump inhibitor release to optimal treatment of NAB. As noted above, Sachs et al. is directed to optimizing combination therapy with an antibiotic for Helicobacter eradication and potential stability advantages of a non-water soluble barrier given the enteric aspect of this formulation.

Formulations disclosed by Sachs et al. are designed to use and/or are specifically disclosed to use enteric coatings. Thus, the formulations of Sachs et al. are designed to release active ingredient when the pH of the digestive track is of a sufficiently high pH.

Still further, attention is directed to pharmaceutical compositions with delayed release of active ingredient for reversible proton pump inhibitors disclosed in Sachs et al., U.S. Pat. No. 6,132,768, the disclosure of which is incorporated by reference herein in its entirety.

WO 01/24777, the disclosure of which is incorporated by reference herein in its entirety, is directed to pharmaceutical compositions, which includes pharmaceutical compositions for multiphase delivery of proton pump inhibitors. WO 01/24777 discusses that current immediate release dosing regimes often result in periods during the day where the intragastric pH is maintained above 3.0, preferably above 4.0, preferably over a 24 hour period is not achieved, and this may become particularly acute during the night where “breakthrough pH” occurs. WO 01/24777 discloses that there is not a constant requirement for the inhibitor because it is postulated that the initial dose inhibits the receptors and it is only when the receptors begin to regenerate that further inhibitor is required. WO 01/24777 discloses that the use of sustained release formulations therefore involves the use of more inhibitor than necessary. Therefore, it is disclosed that it is desirable to provide pulsed release formulations capable of releasing a second dose of inhibitor when the effects of the first dose begin to diminish. WO 01/24777 broadly discloses delayed release of drugs in anticipation of symptoms. However, the only disclosure relative to release of proton pump inhibitor drugs appears to be a delayed release of the proton pump inhibitor in order to delay the release of the proton pump inhibitor for a lengthy period of time, such as 5 or 6 hours or longer (Examples 1 and 3) for apparently release of the active ingredient upon waking, or an immediate release of proton pump inhibitor in a two population formulation (Example 2). Also, WO 01/24777 discloses the use of disintegrants which result in swelling and disintergration of the dosage unit.

US 2002/0160046 A1, the disclosure of which is incorporated by reference herein in its entirety, discloses stabilized formulations containing omeprazole, or a salt thereof, wherein the formulation does not require a separating layer or an enteric release coating. US 2002/0160046 A1 discloses that instead of an enteric coating, the inventive formulation includes a non-enteric time-release (TR) coating applied directly over the omeprazole-containing core. This coating is disclosed to be designed such that the core of the dosage form will rapidly (immediately or catastrophically) disintegrate into an aqueous environment of use when non-acidic media or digestive juice in the environment come into contact with the core. Thus, while US 2002/0160046 A1 discloses that the TR coating generally possesses erosion and/or diffusion properties that are essentially independent of the pH of the external aqueous medium and of the enzymes and bile salts present in the GI tract, it also discloses that the active ingredient is immediately released, and does not appear to disclose sustained release.

WO 00/78293, the disclosure of which is incorporated by reference herein in its entirety, is directed to a a dosage form which is prepared without an enteric coating, and which comprises a core material containing an active ingredient selected from omeprazole, an alkaline salt thereof, S-omeprazole or an alkaline salt thereof, one or more alkaline additives which are alkalizing agents having a pH of not less than 8.5 when measured in a 2% w/w water solution/dispersion with a pH-measuring electrode, and one or more swelling agents. The core is coated with a semipermeable membrane that is able to disrupt or may change its permeability after a predetermined time. WO 00/78293 does not appear to teach or suggest any specific desirable dissolution profile, but appears to indicate that after the pellet formulations have left the stomach, generally within 2-4 hours, the semipermeable membrane covering the individual pellets disrupts and/or starts to release the active ingredient in the small intestine. The sole dissolution profile disclosed in WO 00/78293 is in Example 4 wherein dissolution is measured for 2 hours using 0.1 M HCl and then at pH 6.8. The dissolution profile appears to exemplify a low initial release followed by a rapid release of active ingredient when exposed to the pH 6.8 environment.

In view of the above, there is still an existing need for a proton pump inhibitor formulation that is not pH dependent, such as by relying upon an enteric coating or other pH dependent structure. Moreover, there is still a need for a proton pump inhibitor that can be administered as a preventive and/or therapeutic treatment of NAB which does not require the administration of any other active ingredients, such as histamine₂ receptor antagonists, in conjunction with the proton pump inhibitor. Still further, there is still a need for a proton pump inhibitor formulation that can be administered once-a-day, and optionally two or more times a day, to treat NAB. Moreover, there is a need for a proton pump inhibitor formulation that has a pH independent delayed release of the proton pump inhibitor followed by sustained release of the proton pump inhibitor in a formulation designed to treat NAB. Moreover, there is a need for a formulation that does not require an enteric coating and/or a disintegrant.

### SUMMARY OF THE INVENTION

The present invention relates to proton pump inhibitor formulations.

The present invention also relates to time-based delayed-release, extended-release proton pump inhibitor formulations.

The present invention also relates to pH independent time-based delayed-release, extended-release proton pump inhibitor formulations.

The present invention also relates to pH independent time-based delayed-release, extended-release proton pump inhibitor formulations that can be administered as a preventive and/or therapeutic treatment of NAB.

The present invention also relates to pH independent time-based delayed-release, extended-release proton pump inhibitor formulations that can be administered as a preventive and/or therapeutic treatment of NAB which does not require the administration of any other active ingredients, such as histamine₂ receptor antagonists, in conjunction with the proton pump inhibitor.

The present invention also relates to pH independent time-based delayed-release, extended-release proton pump inhibitor formulations that can be administered once-a-day, and optionally two or more times a day, to treat NAB.

The present invention is directed to a pharmaceutical formulation comprising at least one proton pump inhibitor structured and arranged to provide an initial pH independent time-based delayed-release, and a subsequent extended-release of the at least one proton pump inhibitor, the initial pH independent time-based delayed-release period of the at least one proton pump inhibitor comprising release of at most about 20% of the at least one proton pump inhibitor during a period of about 1 to 4 hours, and the subsequent extended-release of the proton pump inhibitor being over a period of about 3 to 12 hours, and providing an hourly increase in percent release of the at least one proton pump inhibitor during any and all one hour periods of time of less than about 35%.

The present invention is also directed to a proton pump inhibitor formulation, preferably a pH independent time-based delayed-release, extended-release formulation, having a dissolution profile, using a rotating paddle apparatus (USP II) using 900 ml of USP phosphate buffer (pH 6.8) at 37° C. and an agitation speed of 50 rpm of:
2 hours—≦30%,
3 hours—≦60%,
6 hours—≧20%,
8 hours—≧40%, and
12 hours—≧70%.

The present invention is also directed to a method of treating nocturnal acid breakthrough comprising orally administering a pharmaceutical formulation to a mammal, wherein the pharmaceutical formulation comprises at least one proton pump inhibitor structured and arranged to provide an initial pH independent time-based delayed-release, and a subsequent extended-release of the at least one proton pump inhibitor.

The initial pH independent time-based delayed-release of the at least one proton pump inhibitor can comprise release of less than about 10% of the at least one proton pump inhibitor during about 2 to 3 hours, and the subsequent extended-release of the proton pump inhibitor can provide an hourly increase in percent release of the at least one proton pump inhibitor of less than about 30%.

The initial pH independent time-based delayed-release of the at least one proton pump inhibitor can comprise release of less than about 10% of the at least one proton pump inhibitor during about 2 to 3 hours, and the subsequent extended-release of the proton pump inhibitor can provide an hourly increase in percent release of the at least one proton pump inhibitor of less than about 25%.

The initial pH independent time-based delayed-release of the at least one proton pump inhibitor can comprise release of less than about 10% of the at least one proton pump inhibitor during about 2 to 3 hours, and the subsequent extended-release of the proton pump inhibitor can provide an hourly increase in percent release of the at least one proton pump inhibitor of less than about 20%.

The initial pH independent time-based delayed-release of the at least one proton pump inhibitor can comprise no release of the at least one proton pump inhibitor.

The initial pH independent time-based delayed-release of the at least one proton pump inhibitor can comprise release of less than about 5% of the least one proton pump inhibitor.

The initial pH independent time-based delayed-release of the at least one proton pump inhibitor can comprise release of less than about 5% or no release of the at least one proton pump inhibitor for at least about 1 hour.

The initial pH independent time-based delayed-release of the at least one proton pump inhibitor can comprise release of less than about 5% or no release of the at least one proton pump inhibitor for at least about 2 hours.

The initial pH independent time-based delayed-release of the at least one proton pump inhibitor can comprise release of less than about 5% or no release of the at least one proton pump inhibitor for about 2 to 4 hours after administration to a mammal.

The subsequent extended-release of the at least one proton pump inhibitor can comprise release of the at least one proton pump inhibitor over a period of from about 3 to 9 hours.

The subsequent extended-release of the at least one proton pump inhibitor can comprise release of the at least one proton pump inhibitor over a period of from about 4 to 9 hours.

The subsequent extended-release of the at least one proton pump inhibitor can comprise release of the at least one proton pump inhibitor over a period of from about 4 to 6 hours.

The formulation can comprise a diffusion control system. The diffusion control system can comprise a core including the at least one proton pump inhibitor, a diffusion control coating and a pH independent delayed-release coating.

The formulation can comprise a matrix system.

The formulation can comprise an osmotic system.

The formulation can include an insoluble polymer.

The formulation preferably does not include an enteric coating and/or a disintegrant.

The mammal can be a human.

The dissolution profile can also comprise:
2 hours—less than 20%,
3 hours—greater than 10% but less than 30%,
4 hours—greater than 20% but less than 40%,
6 hours—greater than 40% but less than 60%,
8 hours—greater than 60% but less than 80%, and
12 hours—greater than 80%.

The dissolution profile can also comprise:
3 hours—<20%,
4 hours—greater than 10% but less than 30%,
6 hours—greater than 30% but less than 50%,
8 hours—greater than 50% but less than 70%, and
12 hours—greater than 75%.

The dissolution profile can also comprise:
4 hours—<20%,
6 hours—greater than 20% but less than 40%,
8 hours—greater than 40% but less than 60%, and
12 hours—greater than 70%.

The present invention is also directed to methods of treating nocturnal acid breakthrough comprising orally administering formulations according to the present invention to a human.

The present invention is also directed to methods of producing formulations including at least one proton pump inhibitor, comprising including the at least one proton pump inhibitor with pharmaceutical ingredients to provide an initial pH independent time-based delayed-release, and a subsequent extended-release formulations according to the present invention.

The present invention is also directed to formulations including at least one proton pump inhibitor, the formulation having a Tmax of greater than 3.5 hours, preferably greater than about 4 hours, with a preferred Tmax being about 4 to 12 hours. Moreover, the formulations are preferably pH independent time-based delayed-release, extended-release formulations.

### DETAILED DESCRIPTION OF THE INVENTION

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

All percent measurements in this application, unless otherwise stated, are measured by weight based upon 100% of a given sample weight. Thus, for example, 30% represents 30 weight parts out of every 100 weight parts of the sample.

Unless otherwise stated, a reference to a compound or component, includes the compound or component by itself, as well as in combination with other compounds or components, such as mixtures of compounds.

In contrast to current formulations and administration protocols, according to the present invention it has surprisingly been found that proton pump inhibitors can be effectively formulated as controlled release dosage forms without the need for the incorporation of an enteric coating formulated to control release of active ingredient based upon the digestive tract pH conditions. In particular, it has been found that a pH independent time-based (as compared to a pH-based) release mechanism is appropriate to treat various gastric acid related conditions. In particular, a release pattern directed to releasing the proton pump inhibitor after a pH independent delayed period of time, preferably after bed-time, will achieve effective treatment of NAB. Preferably, the delayed period of time for releasing the proton pump inhibitor after oral administration is at least about 1 hour, preferably about 2 hours, with a preferred range being about 2 to 4 hours after bedtime. For example, a typical dosing regimen would include administering the formulation at about 10:00 PM, followed by pH independent time delay for about 2 hours, followed by sustained release to thereby treat expected NAB at about 1:00 AM extending through to about 6:00 AM.

The benefits of the pH independent time-based delayed-release, extended-release proton pump inhibitor formulation of the present invention and the associated release profile are readily apparent to one having ordinary skill in the art following the guidance provided herein. For example, the formulation of the present invention can ensure that the proton pump inhibitor begins its release at the appropriate time. In contrast, an enteric coated formulation cannot guarantee that the active ingredient will be released at the appropriate time. Thus, an enteric coated formulation can be held up in the stomach for differing periods of time. For example, if the enteric coated formulation is held up in the stomach for varying periods of time, the enteric coating would be subjected to uncertainty of release of active ingredient with these varying periods of time. For example, if a formulation is held up in the stomach for over 4 hours, there would not be expected to be release of active ingredient for over 4 hours until the formulation reaches a location in the digestive tract where pH conditions would be suitable for release of the active ingredient.

Still further, there is a subset of people who have non-standard pH environments in their digestive tracts. For example, their lower intestines may not be at a sufficiently high pH to permit release of active ingredient. For example, the pH of the lower intestines may be at a pH as low as 6.4 or lower. In contrast, a pH of 6.5 to 6.8 may be needed to obtain release of the active ingredient due to the pH dependency of the enteric coating. In other words, enteric coated formulations rely upon highly uniform pH conditions from subject to subject. However, as noted above different subjects may have differing pH conditions due to subject to subject variation.

The proton pump inhibitors can comprise any compounds, derivatives of compounds, forms of compounds, such as isomers, stereoisomers, salts, hydrates and solvates, that have activity as proton pump inhibitors. For example, and without limitation proton pump inhibitors according to the present invention include omeprazole, lansoprazole, pantoprazole, rabeprazole, esomeprazole, leminoprazole, tenatoprazole, and their stereoisomers, enantiomers and tautomers, and various salts thereof, such as alkaline salts. Proton pump inhibitors are potent inhibitors of gastric acid secretion, inhibiting H⁺, K⁺-ATPase, the enzyme involved in the final step of hydrogen ion production in the parietal cells. Moreover, the proton pump inhibitors typically include benzimidazole compounds. For example, and without limitation, proton pump inhibitors including various benzimidazole compounds useful in the formulations according to the present invention include those disclosed in the following documents, the disclosures of which are incorporated by reference herein in their entireties: U.S. Pat. Nos. 4,045,563, 4,255,431, 4,182,766, 4,359,465, 4,472,409, 4,508,905, 4,628,098, 4,738,975, 5,045,321, 4,786,505, 4,853,230, 5,045,552, 5,312,824, 5,877,192, 6,207,198, and 6,544,556, EP-A-0295603, EP-A-0166287, EP-A-0519365, EP-A-005129, EP-A-0174726, and GB 2,163,747.

Examples of oral dosage forms according to the present invention include multi-unit and single unit systems and include various mechanisms of release such as diffusion control, osmotic control and matrix control.

Without limitation and solely as an example of the present invention, a particularly preferred example comprises a formulation of the proton pump inhibitor, such as omeprazole, in a single unit diffusion control tablet, where 20 mg omeprazole is compressed into a tablet core with various excipients, and a rate control membrane, preferably a diffusion control membrane, is applied to the core. For example, the rate control membrane can be based on a mixture of a water-insoluble polymer, such as, but not limited to, polyvinyl based polymer, and water soluble materials, such as sugars, including, but not limited to, sucrose, or polyvinylpyrrolidone (PVP). Additionally, pH-independent time delay release coating can be applied externally to the rate control membrane to delay onset of release of the proton pump inhibitor, such as, but not limited to Eudragit® polymers, such as Eudragit® RS and/or Eudragit® RL (Eudragit® is a trade mark of Rohm Pharma polymers a subsidiary of Degussa Corporation), and/or methylcellulose and hydroxypropyl methylcellulose, such as Methocel® which is a product of The Dow Chemical Company, and/or ethylcellulose, such as Ethocel® which is a product of The Dow Chemical Company. A water-soluble finishing coating, such as, but not limited to, OPADRY® WHITE Y-1-7000 and OPADRY® OY/B/28920 WHITE, each of which is available from Colorcon Limited, England, may be further applied.

The release of the at least one proton pump inhibitor according to the present invention is measured using a rotating paddle apparatus (USP II) using 900 ml of USP phosphate buffer (pH 6.8) at 37° C. and an agitation speed of 50 rpm, or an equivalent apparatus and technique.

A preferred release profile of the pH independent time-based delayed-release, extended-release proton pump inhibitor formulation according to the present invention is 2 hours—≦30%, 3 hours—≦60%, 6 hours—≦20%, 8 hours—≦40% and 12 hours—≧70%.

Additional, preferred dissolution profiles include:
(a) 2 hours—less than 20%, 3 hours—greater than 10% but less than 30%, 4 hours—greater than 20% but less than 40%, 6 hours—greater than 40% but less than 60%, 8 hours—greater than 60% but less than 80%, and 12 hours—greater than 80%.
(b) 3 hours—<20%, 4 hours—greater than 10% but less than 30%, 6 hours—greater than 30% but less than 50%, 8 hours—greater than 50% but less than 70%, and 12 hours—greater than 75%.
(c) 4 hours—<20%, 6 hours—greater than 20% but less than 40%, 8 hours—greater than 40% but less than 60%, and 12 hours—greater than 70%.

Expanding upon the above, the pH independent delayed-release, sustained-release formulation according to the present invention may comprise any formulation that permits a pH independent time delay (as compared to a pH-based delay such as which is achieved with an enteric coating) in release of the proton pump inhibitor, and then permits sustained release of the proton pump inhibitor after the pH independent time delay. The pH independent time delayed, sustained release formulations according to the present invention can include any type of formulation that is structured and arranged to provide an initial pH independent time delay of the release of the proton pump inhibitor with subsequent sustained release of the proton pump inhibitor. For example, the sustained release can be obtained by any controlled release obtainable, such as by controlled extended release delivery devices, examples of which are well known to those of ordinary skill in the art. Examples of different formulations are provided in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566, and U.S. patent Publication No. 2003/0118652, the disclosures of which are incorporated herein by reference in their entireties. Suitable components (e.g., polymers, excipients, etc.) for use in controlled release formulations, and methods of producing the same, are also disclosed, e.g., in U.S. Pat. No. 4,863,742, which is incorporated by reference herein in its entirety.

pH independent release according to the present invention can be obtained in any manner that permits the formulation to achieve an initial pH independent time delay followed by sustained release. The two functions can be included in one element of the formulation or can be included in separate elements of the formulation. For example, one preferred formulation, as noted above, includes a diffusion control membrane and a separate pH independent time delay release coating. However, these two coatings can be combined into one coating having both delaying and sustained release functions. Moreover, an osmotic formulation can have a time delaying function combined with the sustained release based on the usually delayed release associated with osmotic formulations.

Examples of pH independent time-based delayed-release, extended-release proton pump inhibitor formulations according to the present invention include, but are not limited to, diffusion-controlled, matrix, osmotic, and ionic exchange systems. These can be in the form of single (monolithic) or multi-unit dosage forms.

With diffusion-controlled extended release dosage forms, the formulation containing the proton pump inhibitor may be surrounded by a semi-permeable membrane. Semi-permeable membranes include those that are permeable to a greater or lesser extent to both water and solute. This membrane may include water-insoluble and/or water-soluble polymers, and are arranged according to the present invention to exhibit pH-dependent or pH-independent solubility characteristics after the initial pH independent time delay. Polymers of these types are described in detail below. Generally, the characteristics of the polymeric membrane (e.g., the composition of the membrane) will determine the nature of release of the proton pump inhibitor.

Sustained controlled release and/or pH independent delayed release can be obtained using Eudragit products, such as EUDRAGIT® RL 30 D which is a highly permeable pH independent polymer for sustained release aqueous formulations, EUDRAGIT® RL PO which is a highly permeable pH independent polymer for matrix formulations, EUDRAGIT® RL 100 which is a highly permeable pH independent polymer insoluble in water, EUDRAGIT® RS 30 D which is a pH independent polymer with low permeability for sustained release aqueous formulations, EUDRAGIT® RS PO which is a pH independent polymer with low permeability for matrix formulations, EUDRAGIT® RS 100 which is a pH independent polymer insoluble in water with low permeability, EUDRAGIT® NE 30 D which is a neutral ester copolymer for wet granulation in sustained release formulations, or EUDRAGIT® NE 40 D which is a neutral ester copolymer with 10% more solids for wet granulation in sustained release formulations. As noted above, Eudragit® is a trade mark of Rohm Pharma polymers a subsidiary of Degussa Corporation. It is noted that Eudragit® products comprise polymethacrylates, such as described in Handbook of Pharmaceutical Excipients, Second Edition, Edited by Wade et al., 1986, pages 362-366, the disclosure of which is incorporated by reference herein in its entirety.

In an osmotic-release system, a selectively permeable membrane encloses a reservoir of the substance of interest, i.e., proton pump inhibitor, at a concentration sufficient to provide an osmotic pressure above a threshold level. Selectively permeable membranes include those that are permeable to water but not to solute. The pore or orifice size of a selectively permeable membrane can be varied so that passage of molecules of the substance through the pore or orifice of the membrane becomes the rate-limiting factor in dispensing the substance into the surrounding environment outside of the dosage form. Alternatively, the reservoir of the substance, in addition to the active ingredient, may also include an inactive substance, such as an osmotic agent, which is present at a concentration sufficient to provide an osmotic pressure above a threshold level. The active substance of interest can be present as a solid or liquid contained within the dosage form. Osmotic devices are particularly suitable for delayed release due to their arrangement and structure to delay release of active ingredient until sufficient pressure is obtained. Moreover, a pH independent time-delay coating can be included with the osmotic device.

Matrix-type systems comprise a proton pump inhibitor mixed with either water-soluble, e.g., hydrophilic polymers, or water-insoluble, e.g., hydrophobic polymers. Generally, the properties of the polymer used in a modified-release dosage form will affect the mechanism of release. For example, the release of the active ingredient from a dosage form containing a hydrophilic polymer can proceed via both surface diffusion and/or erosion. Mechanisms of release from pharmaceutical systems are well known to those skilled in the art. Matrix-type systems can also be monolithic or multiunit, and may be coated with water-soluble and/or water-insoluble polymeric membranes, examples which are described above. Moreover, a coating can be included on the matrix to provide pH independent time delay.

The inventive extended release formulations may rely on ion-exchange resins for the release of the proton pump inhibitor. In such formulations, the drug is bound to ion exchange resin(s) and, when ingested, the release of drug can be determined by the ionic environment within the gastrointestinal tract. Such a formulation can include a pH independent time delay coating.

Depending on the particular need, the inventive formulations may be prepared as tablets, pellets, minitablets, caplets, or any other desired form. Any desired form may be coated or uncoated, and the coating that affects the time delay of the proton pump inhibitor from the formulation is pH-independent providing delay of release of the proton pump inhibitor based upon a pH independent time-delay.

In this regard, without limitation and solely as an example of the present invention, a particularly preferred example comprises a formulation of the proton pump inhibitor, such as omeprazole, in the form of pellets, such as multiparticles or microparticles. In exemplary, non-limiting examples of such an embodiment, pellets can be loaded into capsule form. Thus, a single unit diffusion control capsule can comprise omeprazole in the form of instant release drug loaded multiparticulates, coated with the foregoing polymers to produce modified release and/or delayed/modified release multiparticulate omeprazole formulations loaded into a hard gelatin capsule. In this connection, again without limitation and solely for purposes of illustration, omeprazole may be mixed with a surfactant (e.g. sodium lauryl sulfate) a binder (e.g. polyvinyl pyrollidone or PVP) a glidant (e.g., colloidal silicon dioxide), and antiadherent (e.g., talc) to form a solution or suspension in a suitable solvent in a manner well known to those of ordinary skill in the art. The solution/suspension is then sprayed onto a suitable inert carrier, such as non-pareil seeds and dried to form instant release omeprazole microparticulates. The resultant microparticulates may then be coated with a suitable modified release polymer release system (such as those above and further exemplified in the Examples below) to form modified release and even delayed and/or modified release microparticulates. In this connection, modified and delayed/modified release patterns can be obtained with the microparticulates of the invention using essentially the same techniques and materials as employed in the tablet formulations herein, applied to standard microparticulate technology. The microparticles may be employed in any suitable dosage form.

Pellet dosage forms can be, for example, encapsulated, prepared as a tablet, or administered in a food or drink. One of the advantages of encapsulated pelleted products is that the onset of absorption is less sensitive to stomach emptying. The entrance of the pellets into the small intestine can be more uniform than with non-disintegrating extended-release tablet formulations. In this connection, modified and delayed/modified release patterns can be obtained with the pellets or any other form of the invention using essentially the same techniques and materials as employed in the tablet formulations herein, applied to standard microparticulate technology.

The sustained release of the proton pump inhibitor can be slowed or controlled by using, for example, hydroxypropylmethyl cellulose in varying proportions to provide the desired sustained release profile, other polymer matrices, gels, permeable membranes, osmotic systems, multilayer coatings, microparticles, liposomes, microspheres, or the like, or combinations thereof. Examples of suitable controlled-, delayed-, and/or extended-release formulations are known to those of ordinary skill in the art, and may readily be selected for use with the proton pump inhibitor compositions of the present invention. Thus, tablets, capsules, gelcaps, caplets, and the like, that are adapted for initial pH independent time-delayed release, and subsequent sustained-release, may be used in accordance with the presently disclosed methods. The pH independent delayed-release may be obtained by any materials and/or structures that are pH-independent. However, the sustained-release of the proton pump inhibitor subsequent to the pH independent time delay, while preferably not dependent upon external conditions, may be triggered or stimulated by various inducers, for example pH, temperature, enzymes, water, or other physiological conditions or compounds.

The pH independent time-based delayed-release, sustained-release proton pump inhibitor formulations used in the present methods may include any number of pharmaceutically acceptable excipients. Suitable excipients include, but are not limited to, carriers, such as sodium citrate or dicalcium phosphate; fillers or extenders, such as stearates, silicas, gypsum, starches, lactose, sucrose, glucose, mannitol, talc, or silicic acid; binders, such as hydroxymethyl-cellulose, alginates, gelatin, polyvinyl pyrrolidone, sucrose or acacia; humectants, such as glycerol; disintegrating agents, such as agar, calcium carbonate, potato or tapioca starch, alginic acid, certain silicates, or sodium carbonate; solution retarding agents, such as paraffin; absorption accelerators, such as quaternary ammonium compounds; wetting agents, such as cetyl alcohol or glycerol monostearate; absorbents, such as kaolin and bentonite clay; lubricants, such as talc, calcium stearate, magnesium stearate, solid polyethylene glycols, and sodium lauryl sulfate; stabilizers; coloring agents; buffering agents; dispersing agents; preservatives; and organic bases. The aforementioned excipients are given as examples only and are not meant to include all possible choices. Additionally, many excipients may have more than one role, or be classified in more than one group; the classifications are descriptive only, and not intended to limit any use of a particular excipient.

Examples of suitable organic bases, include, but are not limited to, sodium citrate, sodium succinate, sodium tartrate, potassium citrate, potassium tartrate, potassium succinate, and mixtures thereof. Suitable diluents include, but are not limited to, lactose, talc, microcrystalline cellulose, sorbitol, mannitol, xylitol, fumed silica, stearic acid, magnesium stearate, sodium stearate, and mixtures thereof.

The pH independent time-based delayed-release, extended-release proton pump inhibitor formulation of the present invention is preferably designed as a chronotherapeutic formulation to provide:
(i) a first phase, of time-based, non-release of the proton pump inhibitor, during which there is substantially no release of the proton pump inhibitor, and preferably no release of the proton pump inhibitor. Preferably, at most about 20% of the proton pump inhibitor, more preferably less that about 10%, and even more preferably less than about 5% of the proton pump inhibitor will be released from the formulation during the pH independent time-based delayed-release phase of the at least one proton pump inhibitor. Preferably, the release of the proton pump inhibitor will be time-delayed in order to be released at a time when nocturnal acid breakthrough will occur in the absence of the administration of the formulation according to the present invention. As noted above, this pH independent time delay is preferably about 2 to 4 hours. The first phase is preferably immediately followed by a second phase, wherein:
(ii) a second phase, during which there is a sustained release of the proton pump inhibitor. The proton pump inhibitor will be released over a period of time to maintain the proton pump inhibitor at or above a minimum therapeutic level for a period of from about 3 to 12 hours, more preferably about 4 to 9 hours, even more preferably about 4 to 6 hours, and even more preferably about 5 to 6 hours.

Exemplary formulations according to the present invention include pharmaceutical formulations comprising at least one proton pump inhibitor structured and arranged to provide an initial pH independent time-based delayed-release, immediately followed by a subsequent extended-release of the at least one proton pump inhibitor. The initial pH independent time-based delayed-release period of the at least one proton pump inhibitor comprises release of at most 20%, preferably less than 10%, more preferably less than 5%, and even more preferably no release of the proton pump inhibitor. The pH independent time-based delayed release period preferably comprises a period of about 1 to 4 hours, more preferably about 2 to 3 hours, and preferably about 2 hours. The extended-release of the at least one proton pump inhibitor preferably provides an hourly increase in percent release of the at least one proton pump inhibitor during any and all one hour periods of time of less than about 35%, or less than about 30%, or less than about 25%, or less than about 20%. The extended-release of the at least one proton pump inhibitor preferably is over a time period of about 3 to 12 hours, more preferably over a time period of about 4 to 9 hours, more preferably over a time period of about 4 to 6 hours, and even more preferably over a time period of about 5 to 6 hours. Particularly preferred formulations according to the present invention provide an initial pH independent time-based delayed-release period of the at least one proton pump inhibitor of about 2 to 3 hours releasing less than 10% of the proton pump inhibitor, the extended-release of the at least one proton pump inhibitor providing an hourly increase in percent release of the at least one proton pump inhibitor during any and all one hour periods of time of less than about 25%, with the extended-release of the at least one proton pump inhibitor preferably being over a time period of about 4 to 6 hours.

When the expression initial is utilized herein, the expression does not exclude the formulation including ingredient(s) associated therewith which can be released in conjunction with the period of delay. Thus, the formulation can include any materials associated therewith that can be released during the initial pH independent delayed-release period. For example, and without limitation, a coating can be included on the formulation according to the present invention to release a material such as a flavoring agent and/or can include an esthetically pleasing layer.

Moreover, the expression “an hourly increase in percent release of the at least one proton pump inhibitor during any and all one hour periods of time” indicates the following. During the sustained release phase, any measurement over any one hour period of time will show a release of less than about 35%, or less than about 30%, or less than about 25%, or less than about 20% of the at least one proton pump inhibitor.

Expanding upon the above, if about 5% of the at least one proton pump inhibitor is released during a measured period of time, for example during the first hour of extended release, than at most a total of about 40% (5% plus 35%) of the at least one proton pump inhibitor, more preferably at most a total of about 35% (5% plus 30%) of the at least one proton pump inhibitor, more preferably at most a total of about 30% (5% plus 25%), and most preferably at most about 25% (5% plus 20%) of the at least one proton pump inhibitor will be released over a total of the first 2 hours of sustained release.

The sustained release of the at least one proton pump inhibitor can be in any manner according to the present invention as long as the release is over the period of time and at a maximum amount as noted above. Thus, the release can be linear or substantially linear, but can be in any manner over the sustained-release time period. For example, a release profile can include an hourly release of about 20% of the at least one proton pump inhibitor over a period of about 5 hours, or a maximum hourly release of about 15 to 30% percent release of the at least one proton pump inhibitor hourly over a period of about 4 to 6 hours.

Such formulations preferably provide a pH independent time delay in release of the proton pump inhibitor while the subject is sleeping. The time-delay can be formulated to release a subtherapeutic level of the proton pump inhibitor, and preferably no or substantially no release of the proton pump inhibitor during an initial time period, and subsequent sustained release of therapeutic concentrations, preferably during sleeping of the patient. However, the patient can be awake when the proton pump inhibitor begins its sustained release.

The pH independent time-based delayed-release, extended-release proton pump inhibitor of the present invention can be administered as a once-a-day oral formulation at night, preferably about 9 to 11 PM, and even more preferably about 10 PM.

The pH independent time-based delayed-release, extended-release proton pump inhibitor formulation according to the present invention can be administered in combination with other therapies, such as, but not limited to, histamine₂ receptor antagonists. Moreover, the pH independent time-based delayed-release, extended-release proton pump inhibitor formulation can be administered to a patient once-a-day, or greater than once a day, such as twice-a-day.

The pH independent time-based delayed-release, extended-release proton pump inhibitor formulations of the present invention can comprise at least one polymeric material, such as previously noted. Suitable water-soluble polymers include, but are not limited to, polyvinyl alcohol, polyvinylpyrrolidone, methylcellulose, hydroxypropylcellulose, hydroxypropylmethyl cellulose or polyethylene glycol, and/or mixtures thereof. Suitable water-insoluble polymers include, but are not limited to, ethylcellulose, cellulose acetate cellulose propionate, cellulose acetate propionate, cellulose acetate butyrate, cellulose acetate phthalate, cellulose triacetate, poly (methyl methacrylate), poly (ethyl methacrylate), poly (butyl methacrylate), poly (isobutyl methacrylate), poly (hexyl methacrylate), poly (isodecyl methacrylate), poly (lauryl methacrylate), poly (phenyl methacrylate), poly (methyl acrylate), poly (isopropyl acrylate), poly (isobutyl acrylate), poly (octadecyl acrylate), poly (ethylene), low density poly (ethylene), high density poly (ethylene), poly (ethylene oxide), poly (ethylene terephthalate), poly (vinyl isobutyl ether), poly (vinyl acetate), poly (vinyl chloride) or polyurethane, and/or mixtures thereof.

As the present formulation is a pH independent time-based, delayed-release, extended-release proton pump inhibitor formulation, the formulation does not include an enteric coating. However, enteric polymers can be included in the formulation as long as the enteric polymers do not affect the pH independent time-based delayed-release of the proton pump inhibitor formulations of the present invention. For example, enteric polymers can be included in an inner portion of the formulation, e.g., a portion of the formulation that does not provide pH independent time delay, and/or at a concentration so as not to provide pH dependency when included in an outer portion of the formulation.

The core may comprise a polymeric material comprising a major proportion (i.e., greater than 50% of the total polymeric content) of one or more pharmaceutically acceptable water-soluble polymers, and optionally a minor proportion (i.e., less than 50% of the total polymeric content) of one or more pharmaceutically acceptable water insoluble polymers.

Alternatively, the core may comprise a polymeric material comprising a major proportion (i.e., greater than 50% of the total polymeric content) of one or more pharmaceutically acceptable water insoluble polymers, and optionally a minor proportion (i.e., less than 50% of the total polymeric content) of one or more pharmaceutically acceptable water-soluble polymers. The formulations may optionally contain a coating membrane partially or completely surrounding the core, comprising a major proportion of one or more pharmaceutically acceptable film-forming, water-insoluble polymers, and optionally a minor proportion of one or more pharmaceutically acceptable film-forming, water-soluble polymers. The water-insoluble polymer may form an insoluble matrix having a high or low permeability to the proton pump inhibitor

The polymeric material typically comprises one or more soluble excipients so as to increase the permeability of the polymeric material. Suitably, the soluble excipient is selected from among a soluble polymer, a surfactant, an alkali metal salt, an organic acid, a sugar, and a sugar alcohol. Such soluble excipients include polyvinyl pyrrolidone, polyethylene glycol, sodium chloride, surfactants such as sodium lauryl sulfate and polysorbates, organic acids such as acetic acid, adipic acid, citric acid, fumaric acid, glutaric acid, malic acid, succinic acid, and tartaric acid and sugars such as dextrose, fructose, glucose, lactose and sucrose, and sugar alcohols such as lactitol, maltitol, mannitol, sorbitol and xylitol, xanthan gum, dextrins, and maltodextrins. In some particular embodiments, polyvinyl pyrrolidone, mannitol and/or polyethylene glycol are the soluble excipients. The soluble excipient is typically used in an amount of from about 0.5% to about 80% by weight, based on the total dry weight of the polymer.

The polymeric material can also include one or more auxiliary agents such as a filler, a plasticizer and/or an anti-foaming agent. Representative fillers include talc, fumed silica, glyceryl monostearate, magnesium stearate, calcium stearate, kaolin, colloidal silica, gypsum, micronized silica and magnesium trisilicate. The quantity of filler used typically ranges from about 0.5% to about 300% by weight, and can range from about 0.5% to about 100%, based on the total dry weight of the polymer. In one embodiment, talc is the filler.

The coatings can also include a material that improves the processing of the polymers. Such materials are generally referred to as plasticizers and include, for example, adipates, azelates, benzoates, citrates, isoebucates, phthalates, sebacates, stearates and glycols. Representative plasticizers include acetylated monoglycerides, butyl phthalyl butyl glycolate, dibutyl tartrate, diethyl phthalate, dimethyl phthalate, ethyl phthalyl ethyl glycolate, glycerin, ethylene glycol, propylene glycol, triacetin citrate, triacetin, tripropinoin, diacetin, dibutyl phthalate, acetyl monoglyceride, polyethylene glycols, castor oil, triethyl citrate, polyhydric alcohols, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, glyceryl monocaprylate, and glyceryl monocaprate. In one embodiment, the plasticizer is dibutyl sebacate. The amount of plasticizer used in the polymeric material typically ranges from about 0.5% to about 50%, for example, about 0.5, 1, 2, 5, 10, 20, 30, 40, or 50%, based on the weight of the dry polymer.

In one embodiment, the anti-foaming agent is simethicone. The amount of anti-foaming agent used typically comprises from about 0% to about 0.5% of the final formulation.

The amount of polymer to be used in the time-based delayed-release, extended-release proton pump inhibitor formulations of the present invention can be adjusted to achieve the desired drug delivery properties, including the amount of drug to be delivered, that rate and location of drug delivery, the time delay of drug release, and the size of the multiparticulates in the formulation. The amount of polymer applied typically provides about a 0.5% to about 100% weight gain to the cores. In one embodiment, the weight gain from the polymeric material is about 2% to about 70%.

The combination of all solid components of the polymeric material, including co-polymers, fillers, plasticizers, and optional excipients and processing aids, typically provides an about 0.5% to about 450% weight gain on the cores. In one embodiment, the weight gain is about 2% to about 160%.

The polymeric material can be applied by any known method, for example, by spraying using a fluidized bed coater (e.g., Wurster coating) or pan coating system.

The coated cores are typically dried or cured after application of the polymeric material. Curing means that the multiparticulates are held at a controlled temperature for a time sufficient to provide stable release rates. Curing can be performed for example in an oven or in a fluid bed drier. Curing can be carried out at any temperature above room temperature.

A sealant or barrier can be applied to the polymeric coating. A sealant or barrier layer may also be applied to the core prior to applying the polymeric material. The sealant or barrier layer does not modify the release of the proton pump inhibitor. Suitable sealants or barriers are permeable or soluble agents such as hydroxypropyl methylcellulose, hydroxypropyl cellulose, hydroxypropyl ethylcellulose, and xanthan gum. Hydroxypropyl methylcellulose is particularly useful in this regard.

Other agents can be added to improve the processability of the sealant or barrier layer. Such agents include talc, colloidal silica, polyvinyl alcohol, titanium dioxide, micronized silica, fumed silica, glycerol monostearate, magnesium trisilicate or magnesium stearate or a mixture thereof. The sealant or barrier layer can be applied from solution (e.g., aqueous) or suspension using any known means, such as a fluidized bed coater (e.g., Wurster coating) or pan coating system. Suitable sealants or barriers include, for example, OPADRY® WHITE Y-1-7000 and OPADRY® OY/B/28920 WHITE, each of which is available from Colorcon Limited, England.

The pH independent time-based delayed-release, extended-release proton pump inhibitor formulations of the present invention can be in the form of caplets, capsules, particles for suspension prior to dosing, sachets, or tablets. When the dosage form is in the form of tablets, the tablets may be, for example, disintegrating tablets, fast dissolving tablets, effervescent tablets, fast melt tablets, and/or mini-tablets. The dosage form can be of any shape suitable for oral administration of a drug, such as spheroidal, cube-shaped oval, or ellipsoidal. The dosage forms will be prepared from the multiparticulates in a manner known in the art and include addition pharmaceutically acceptable excipients, as desired.

The thickness of the polymer in the formulations, the amounts and types of polymers, and the ratio of water-soluble polymers to water-insoluble polymers in the controlled release formulations are generally selected to achieve a desired release profile of the proton pump inhibitor. For example, by increasing the amount of water insoluble-polymer relative to the water soluble-polymer, the release of the drug may be delayed or slowed.

The invention is further illustrated by reference to the following examples. It will be apparent to those skilled in the art that many modifications, both to the materials and methods, may be practiced without departing from the purpose and scope of the invention.

Without further elaboration, it is believed that one skilled in the art can, using the preceding description, utilize the present invention to its fullest extent.

The following preferred specific embodiments are, therefore, to be construed as merely illustrative, and not limitative of the remainder of the disclosure in any way whatsoever.

### EXAMPLES

The following prophetic examples are provided to provide even further guidance with respect to the making and using of the present invention.

### Example 1

This example is directed to proton pump inhibitor delayed onset diffusion controlled membrane coated tablets which include an instant release formulation, a diffusion controlled membrane coating, and a delayed onset release coating.

(A) Instant Release Core Formulations

Instant release core formulations can be prepared from the following exemplary, non-limiting, formulations as depicted in Table 1:

TABLE 1
Instant Release Core Formulations

| | | Qty | Qty | Qty | Qty |
|---|---|---|---|---|---|
| Ingredient | FUNCTION | % (w/w) | % (w/w) | % (w/w) | % (w/w) |
| PPI | Active | 20.00 | 20.00 | 20.00 | 20.00 |
| LACTOSE ANHYDROUS | Diluent | 69.50 | 57.13 | 44.75 | 22.37 |
| (DIRECT COMPRESSION | | | | | |
| GRADE) | | | | | |
| MICROCRYSTALLINE | Dry Binder/ | 10.00 | 22.37 | 34.75 | 57.13 |
| CELLULOSE | diluent | | | | |
| (AVICEL PH200) | | | | | |
| MAGNESIUM | Lubricant | 0.5 | 0.5 | 0.5 | 0.5 |
| STEARATE | | | | | |
| TOTAL | | 100.00 | 100.00 | 100.00 | 100.00 |

(B) Tablet Manufacturing Process

Instant release core formulations can be prepared from the following exemplary, non-limiting, production technique:

1. Weigh the ingredients using a suitable balance.

2. Add the ingredients, except Magnesium Stearate to a V type blender.

3. Mix for 30 minutes (until a homogeneous blend is produced).

4. Add the Magnesium Stearate to the blender.

5. Mix for a Further 5 minutes

6. Compress into tablets (100 mg weight) on a suitable tablet machine.

Tablet Weight 100 mg for 20 mg strength

(C) Diffusion Control Membrane Coating Formulation

Diffusion control membrane coating formulations can be prepared from the following exemplary, non-limiting, formulations depicted in Table 2 by combining the indicated ingredients:

TABLE 2
Diffusion Control Membrane Coating Formulation

| | Formula A | Formula B | Formula C | Formula D |
|---|---|---|---|---|
| Ingredient | mg/tab | mg/tab | mg/tab | mg/tab |
| POLYMER¹ | 11.00 | 9.20 | 11.00 | 4.24 |
| SUCROSE | 29.00 | 17.00 | 21.00 | 15.7 |
| ACETYL TRIBUTYL | 2.00 | 1.60 | 1.90 | 0.48 |
| CITRATE | | | | |
| CASTOR OIL | 1.00 | 1.2 | 1.4 | 0.37 |
| POLYMERIZED | | | | |
| SODIUM HYDROGEN | 1.00 | 1.00 | 1.00 | 0.58 |
| CARBONATE | | | | |
| ACETONE² | N/A | N/A | N/A | N/A |
| TOTAL | 44.0 | 30.0 | 36.3 | 21.37 |

¹= Polymer is a terpolymer of polyvinyl chloride, polyvinyl acetate and polyvinyl alcohol (terpolymer PVC/PVAc/PVOH)
²= Solvent is removed during processing.

(D) Manufacturing Process.

Diffusion control membrane coating formulations can be prepared from the following exemplary, non-limiting, production technique:

1. Load the tablets prepared in (B) into a suitable coating machine, e.g., Glatt, Acelacota.

2. Spray the Polymer coating solution onto the tablets.

3. Once the required amount of polymer coating solution has been applied, dry the tablets in a coating machine.

(E) Delayed Onset Release Coating

The above membrane coated tablets in (D) can be coated with the alternative pH independent polymer coatings (Polymer Systems A,B or C) described in Tables 3, 4 and 5, respectively:

TABLE 3
Polymer System A

| Ingredient | FUNCTION | (Batch g) |
|---|---|---|
| ETHOCEL ® | Controlled Release Polymer | 760.00 |
| METHOCEL ® | Controlled Release Polymer | 330.0 |
| ACETYL | Plasticizer | 220.00 |
| TRIBUTYL | | |
| CITRATE | | |
| ETHANOL | Solvent | 2680 |
| WATER | Solvent | 451 |
| TOTAL | | 4441 |

TABLE 4
Polymer System B

| Ingredient | FUNCTION | (Batch g) |
|---|---|---|
| EUDRAGIT ® RS 12.5 | Delayed onset | 900.00 |
| | Release Polymer | |
| EUDRAGIT ® RL | Delayed onset | 300.00 |
| 12.5 | Release Polymer | |
| TALC | Antiadherent | 105.00 |
| DIBUTYL | Plasticizer | 15.00 |
| SEBECATE | | |
| MAGNESIUM | Antiadherant | 30.00 |
| STEARATE | | |
| ACETONE | Solvent | 825.00 |
| ISOPROPYL | Solvent | 825 |
| ALCOHOL | | |
| TOTAL | | 3000.00 |

TABLE 5
Polymer System C

| Ingredient | FUNCTION | (Batch g) |
|---|---|---|
| EUDRAGIT ® RS 30D | Delayed onset | 200.00 |
| | Release | |
| | Polymer | |
| TALC | Antiadherent | 60.00 |
| TRIETHYL | Plasticizer | 12.00 |
| CITRATE | | |
| SIMETHICONE | Dispersant | 1.00 |
| EMULSION | | |
| WATER | Solvent | 392.00 |
| TOTAL | | 665.00 |

(F) Manufacturing Process.

1. Load the tablets into a suitable coating machine (e.g Glatt, Acelacota).

2. Spray the Polymer coating solution on to the tablets.

3. Once the required amount of polymer coating solution has been applied, dry the tablets in coating machine.

### Example 2

(A) Matrix Formulations

Modified Release Tablet formulations of proton pump inhibitors using different concentrations of Methocel® (Hydroxypropylmethylcellulose) can be prepared from the following exemplary, non-limiting, formulations as depicted in Table 6.

TABLE 6
Matrix Tablet Formulations

| | | Qty % | Qty % | Qty % |
|---|---|---|---|---|
| Ingredient | FUNCTION | (w/w) | (w/w) | (w/w) |
| PPI | Active | 20.00 | 20.00 | 20.00 |
| LACTOSE | Diluent | 20.58 | 15.78 | 10.00 |
| AVICEL ® | Dry Binder | 33.72 | 18.52 | 4.30 |
| PH101 | diluent | | | |
| METHOCEL ® | Controlled | 20.00 | 40.00 | 60.00 |
| | Release | | | |
| | Polymer | | | |
| COLLOIDAL | Glidant | 0.20 | 0.20 | 0.20 |
| SILICON | | | | |
| DIOXIDE | | | | |
| MAGNESIUM | Lubricant | 0.50 | 0.50 | 0.50 |
| STEARATE | | | | |
| PVP | Binder | 5.0 | 5.0 | 5.0 |
| *ISOPROPYL | Solvent | N/A | N/A | N/A |
| ALCOHOL | | | | |
| TOTAL | | 100 | 100 | 100 |

*Removed during processing.

Various grades of Methocel® can also be used, e.g. K, E, Series as described by the material supplier (Dow Chemicals).

(B) Tablet Production

WET GRANULATION PROCESS (Using Formulation Above in Table 6)

1. Weigh Ingredients

2. Dissolve the PVP in the IPA

3. Place PPI, Methocel, 50% Avicel, 50% Lactose, in a suitable mixer. (Planetary (Hobart), High Shear(Diosna/Fielder).

4. Mix for 15 minutes to produce a homogenous mix.

5. Continue mixing and add to the mixture the granulating fluid (PVP Solution).

6. Mix until a suitable granulation end point is achieved (add more IPA if needed to produce a suitable granule).

7. Dry the granules (oven or fluidization equipment ) until acceptable level of moisture (<1.0 wt %) and IPA (<0.5 wt %) is achieved.

8. Pass the dry granulate through a suitable comminution equipment (Co-Mill, Fitzpatrick mill) fitted with a suitable sized screen (100-500 micron)

9. Place granulate produced in 9, in a blender add, Colloidal Silicon Dioxide, and the remainder of the Lactose and Avicel®.

10. Mix for 15 minutes.

11. Add the Magnesium Stearate and mix for further 5 minutes.

12. Compress into tablets on a suitable tablet machine.

Or

DIRECT COMPRESSION PROCESS (Using Formulation Above in Table 6)

1. Weigh Ingredients

2. Place all ingredients (except Magnesium Stearate) into a suitable blender (V or Y type).

3. Mix for 15 minutes until homogeneous.

4. Add the Magnesium Stearate

5. Mix for a further 5 minutes

6. Compress tablet blend into oval tablets.

(C) Delayed Release Coating

The above tablets can be coated with pH independent polymer coatings in the manner described above in Example 1.

### Example 3

Release Testing of Delayed Onset Release Tablets

Since these tablets are designed to achieve a release profile independent of pH, with the characteristics of an initial delayed release/onset followed by an extended release phase the testing is carried out at a single pH medium condition. Since PPI's are subject to degradation at lower pH values, it is preferred to conduct the release testing at pH 6.8 or higher. However, other pH's can be utilized.

Test conditions involve testing the release in a rotating paddle apparatus (USP II) using 900 ml of USP phosphate buffer (pH 6.8) at 37° C. and an agitation speed of 50 R.P.M.

Samples are taken from the release test vessel at predetermined times to characterize the release profile.

The following release profiles can result from the tablets coated with the alternative delayed onset polymer systems A, B or C from Example 1.

TABLE 6A
(% Released)

| Time (hours) | System A | System B | System C |
|---|---|---|---|
| 1 | 0 | 0 | 0 |
| 2 | 10 | 0 | 0 |
| 3 | 20 | 10 | 0 |
| 4 | 30 | 20 | 10 |
| 6 | 50 | 40 | 30 |
| 8 | 70 | 60 | 50 |
| 12 | 90 | 85 | 80 |

If tested in medium buffered at lower pH values similar profile characteristics will be achieved, although a correction will be required to account for the degradation of the PPI at that pH value.

### Example 4

Clinical Testing of Delayed Onset Tablets Based on Alternative Polymer Systems A,B and C

For a total of 12 GERD patients in testing. Baseline gastric pH measurements are made with patients off all acid secretion related medication. Subsequent pH measurements are made at the end of the following 4 treatment regimens
1) Commercial Omeprazole Reference(as Prilosec) 20 mg daily at 10:00 PM for 2 weeks
2) Delayed onset ER Tablet Polymer System A 20 mg daily at 10:00 PM for 2 weeks
3) Delayed onset ER Tablet Polymer System B 20 mg daily at 10:00 PM for 2 weeks
4) Delayed onset ER Tablet Polymer System C 20 mg daily at 10:00 PM for 2 weeks

A pH probe is placed 10 cm below the manometrically identified lower esophageal sphincter and intragastric pH is recorded from 10:00 PM until 8:00 AM the next morning.

The median percentage of time with intragastric pH<4 and 3 are estimated. Also median hourly intragastric pH is calculated. Acid breakthrough is defined as intragastric pH<4 for more than 1 hour.

Treatments are compared with baseline and with each other. In particular the benefits of the delayed onset test tablets is demonstrated relative to the commercial reference product.

### Example 5

Biostudy

An open label, single dose, 4-treatment, four period, balanced, randomized, crossover study is designed to compare and assess the relative bioavailability of the three delayed onset formulations with a commercial reference product (Prilosec). The test formulations are as described above, i.e., based on the alternative Polymer Systems A,B,C.

Sixteen healthy volunteers are dosed on each of four occasions with at least a seven-day washout period between each dose. Dosing occurs at 10:00 PM after at least a 4 hour fast. Water is proscribed for one hour before and one hour after dosing except for the 150 mL of water at the time of dosing. Venous blood samples are obtained at regular time intervals immediately prior to and following each dosing for a period of up to 48 hours. Concentrations of Omeprazole in plasma are measured by HPLC. Individual plasma concentration curves are constructed and individual, mean, and relative pharmacokinetic parameters are estimated including Tmax, Cmax and AUC.

Whereas the reference product will show a Tmax of 0.5-3.5 hours, the test products will show a significantly delayed Tmax with values >3.5 hours, preferably greater than about 4 hours, with a preferred range being about 4-12 hours. In addition, the test products will show significantly lower Cmax values and more extended plasma concentrations at later time points.

### Example 6

This example is directed to modified release formulations of proton pump inhibitors in microparticle form.

(A) Instant Release Drug Loaded Multiparticulates

Instant release microparticle formulations can be prepared from the following, non-limiting, exemplary formulations depicted in Table 7:

TABLE 7

| | | Qty | Qty | Qty | Qty |
|---|---|---|---|---|---|
| Ingredient | FUNCTION | (mg) | (mg) | (mg) | (mg) |
| Omeprazole | Active | 20.00 | 20.00 | 20.00 | 20.00 |
| NON PAREIL | Inert carrier | 120.00 | 120.00 | 120.00 | 120.00 |
| SEEDS | | | | | |
| PVP | Binder | 2.50 | 2.50 | 5.00 | 10.00 |
| TALC) | Antiadherent | 5.00 | 5.00 | 10.00 | 15.00 |
| COLLOIDAL | Glidant | 2.50 | 2.50 | 5.00 | 7.00 |
| SILICON DIOXIDE | | | | | |
| SODIUM LAURYL | Surfactant | — | 0.75 | 1.50 | 2.50 |
| SULPHATE | | | | | |
| SUITABLE | Solvent | N/A | N/A | N/A | N/A |
| SOLVENT* | | | | | |
| TOTAL | | 150.00 | 150.75 | 161.50 | 174.50 |

*Removed during processing

Manufacturing Process—Drug Loaded Instant Release Multiparticulates

Drug loaded, instant release microparticles can be prepared using the following exemplary, non-limiting, production technique:

1. The Omeprazole, surfactant, binder, glidant and antiadherent is dissolved/suspended in a suitable solvent.

2. The solution suspension is then sprayed on to the non-pareil particles (sugar spheres) using an appropriate fluidized coating machine (e.g Glatt, Acelacota).

3. Once all the solution suspension has been applied on to the Non Pareil Seeds the drug loaded instant release multiparticulates are dried in the fluidized coating machine.

Modified Release Dosage Form:

These Instant release Omeprazole multiparticulates can then be coated with a number of different modified release polymer systems such as those described below to produce a modified release Omeprazole dosage form.

(B) Modified Release Multiparticulate Formulation of Proton Pump Inhibitors

A modified release multiparticulate formulation can be prepared in accordance with the following, non-limiting, exemplary embodiment.

Modified Release Multiparticulates

Modified release microparticles can be prepared using the formulation outlined below:

Polymer System A

A polymer solution (“a”) may be prepared, using the formulation of table 8 as follows:

TABLE 8

| | | (Batch |
|---|---|---|
| Ingredient | FUNCTION | g) |
| EUDRAGIT RS 30D | Controlled | 200.00 |
| | Release | |
| | Polymer | |
| TALC | Antiadherent | 60.00 |
| TRIETHYL | Plasticizer | 12.00 |
| CITRATE | | |
| SIMETHICONE | Dispersant | 1.00 |
| EMULSION | | |
| WATER | Solvent | 392.00 |
| TOTAL | | 665.00 |

Modified Release Multiparticulate Formulation of Proton Pump Inhibitor Using Polymer solution A

A modified release microparticle formulation using polymer solution A may be prepared in accordance with the following, non-limiting, exemplary formulation depicted in Table 9, below and the following, non-limiting, exemplary procedure which follows it.

TABLE 9

| | Batch | Batch | Batch | Batch | Batch |
|---|---|---|---|---|---|
| Ingredient | (g) | (g) | (g) | (g) | (g) |
| Omeprazole | 1000 | 1000 | 1000 | 1000 | 1000 |
| Loaded | | | | | |
| Instant Release | | | | | |
| Multiparticulates | | | | | |
| *Polymer | 50 | 100 | 200 | 250 | 300 |
| Solution A | | | | | |
| TOTAL | 1050 | 1100 | 1200 | 1250 | 1300 |
| *This represents the amount of solid content in the polymer solution A as the water is removed during processing. The amount of solids applied can be adjusted depending on | | | | | |
| # the type of dissolution profile that is required. Increased amounts of polymer solids will produce decreasing dissolution profiles. | | | | | |

Manufacturing Process—Modified Release Omeprazole Formulation

1. Load the drug loaded instant release mutiparticulates in to a suitable fluidized coating machine (e.g Glatt).

2. Spray the Polymer coating solution on to the drug loaded instant release multiparticulates.

3. Once the required amount of polymer coating solution has been applied, dry the product in the fluidized coating machine.

4. Encapsulate the product in a hard gelatin capsule using an automated encapsulation machine, sufficient to obtain 20 mg dose of Omeprazole per capsule.

(C) Modified Release Multiparticulate Formulation of Proton Pump Inhibitors

Yet another modified release multiparticulate formulation can be prepared in accordance with the following, non-limiting, exemplary embodiment.

Modified Release Multiparticulates

Modified release microparticles can be prepared using the formulation outlined below:

Polymer System B

A polymer solution (“b”) may be prepared, using the formulation of table 10 as follows:

TABLE 10

| | | Batch |
|---|---|---|
| Ingredient | FUNCTION | (g) |
| EUDRAGIT RS 12.5 | Controlled | 900.00 |
| | Release | |
| | Polymer | |
| EUDRAGIT RL 12.5 | Controlled | 300.00 |
| | Release | |
| | Polymer | |
| TALC | Antiadherent | 105.00 |
| DIBUTYL SEBECATE | Plasticizer | 15.00 |
| MAGNESIUM | Antiadherant | 30.00 |
| STEARATE | | |
| ACETONE | Solvent | 825.00 |
| ISOPROPYL | Solvent | 825 |
| ALCOHOL | | |
| TOTAL | | 3000.00 |

Modified Release Multiparticulate Formulation of Proton Pump Inhibitor Using Polymer Solution B.

A modified release microparticle formulation using polymer solution B may be prepared in accordance with the following, non-limiting, exemplary formulation depicted in Table 11, below and the following, non-limiting, exemplary procedure which follows it.

TABLE 11

| | Batch | Batch | Batch | Batch | Batch |
|---|---|---|---|---|---|
| Ingredient | (g) | (g) | (g) | (g) | (g) |
| Omeprazole | 1000 | 1000 | 1000 | 1000 | 1000 |
| Loaded | | | | | |
| Instant Release | | | | | |
| Multiparticulates | | | | | |
| *Polymer | 50 | 100 | 200 | 300 | 400 |
| Solution B | | | | | |
| TOTAL | 1050 | 1100 | 1200 | 1300 | 1400 |
| *This represents the amount of solid content in the polymer solution A as the solvent is removed during processing. The amount of solids applied can be adjusted depending on | | | | | |
| # the type of dissolution profile that is required. Increased amounts of polymer solids will produce decreasing dissolution profiles. | | | | | |

Manufacturing Process—Modified Release Proton Pump Inhibitor Formulation

1. Load the drug loaded instant release mutiparticulates in to a suitable fluidized coating machine (e.g Glatt).

2. Spray the Polymer coating solution on to the drug loaded instant release multiparticulates.

3. Once the required amount of polymer coating solution has been applied, dry the product in the fluidized coating machine.

4. Encapsulate the product in a hard fluidizede capsule using an automated encapsulation machine, sufficient to obtain 20 mg dose of Omeprazole per capsule.

Modified Release Multiparticulate Formulation of Proton Pump Inhibitor Using Polymer Solution C

A modified release microparticle formulation using polymer solution C may be prepared in accordance with the following, non-limiting, exemplary formulation depicted in Table 12, below and the following, non-limiting, exemplary procedure which follows it.

TABLE 12

| Ingredient | FUNCTION | (Batch g) |
|---|---|---|
| METHOCEL | Modified | 750.00 |
| | release | |
| | Polymer | |
| ETHOCEL | Modified | 250.0 |
| | release | |
| | Polymer | |
| TRIETHYL CITRATE | Plasticizer | 12.00 |
| Isopropyl Alcohol (IPA) | Solvent | 2000 |
| TOTAL | | 3012 |

Modified Release Multiparticulate Formulation of Proton Pump Inhibitor using Polymer Solution C

A modified release microparticle formulation using polymer solution C may be prepared in accordance with the following, non-limiting, exemplary formulation depicted in Table 13, below and the following, non-limiting, exemplary procedure which follows it.

TABLE 13

| | Batch | Batch | Batch | Batch | Batch |
|---|---|---|---|---|---|
| Ingredient | (g) | (g) | (g) | (g) | (g) |
| Omeprazole | 1000 | 1000 | 1000 | 1000 | 1000 |
| Loaded | | | | | |
| Instant Release | | | | | |
| Multiparticulates | | | | | |
| *Polymer | 50 | 100 | 200 | 300 | 400 |
| Solution C | | | | | |
| TOTAL | 1050 | 1100 | 1200 | 1300 | 1400 |
| *This represents the amount of solid content in the polymer solution as the solvent is removed during processing. The amount of solids applied can be adjusted depending on | | | | | |
| # the type of dissolution profile that is required. Increased amounts of polymer solids will produce decreasing dissolution profiles. | | | | | |

Manufacturing Process—Modified Release Proton Pump Inhibitor Formulation

1. Load the drug loaded instant release mutiparticulates in to a suitable fluidized coating machine (e.g Glatt).

2. Spray the Polymer coating solution on to the drug loaded instant release multiparticulates.

3. Once the required amount of polymer coating solution has been applied, dry the product in the fluidized coating machine.

4. Encapsulate the product in a hard fluidizede capsule using an automated encapsulation machine, sufficient to obtain 20 mg dose of Omeprazole per capsule.

Modified Release Multiparticulate Formulation of Proton Pump Inhibitor Using Polymer Solution D

A modified release microparticle formulation using polymer solution D may be prepared in accordance with the following, non-limiting, exemplary formulation depicted in Table 14, below and the following, non-limiting, exemplary procedure which follows it.

TABLE 14

| | | (Batch |
|---|---|---|
| Ingredient | FUNCTION | Kg) |
| ETHOCEL E7 | Controlled | 1.730 |
| | Release | |
| | Polymer | |
| PVP K30 | Controlled | 0.073 |
| | Release | |
| | Polymer | |
| TALC | Antiadherent | 1.500 |
| ACETONE | Solvent | 28.00 |
| TOTAL | | 31.303 |

Modified Release Multiparticulate Formulation of Proton Pump Inhibitor Using Polymer Solution D

A modified release microparticle formulation using polymer solution D may be prepared in accordance with the following, non-limiting, exemplary formulation depicted in Table 15, below and the following, non-limiting, exemplary procedure which follows it.

TABLE 15

| | Batch | Batch | Batch | Batch | Batch |
|---|---|---|---|---|---|
| Ingredient | (g) | (g) | (g) | (g) | (g) |
| Omeprazole | 1000 | 1000 | 1000 | 1000 | 1000 |
| Loaded | | | | | |
| Instant Release | | | | | |
| Multiparticulates | | | | | |
| *Polymer | 50 | 100 | 200 | 300 | 400 |
| Solution D | | | | | |
| TOTAL | 1050 | 1100 | 1200 | 1300 | 1400 |
| *This represents the amount of solid content in the polymer solution as the solvent is removed during processing. The amount of solids applied can be adjusted depending on | | | | | |
| # the type of dissolution profile that is required. Increased amounts of polymer solids will produce decreasing dissolution profiles. | | | | | |

Manufacturing Process—Modified Release Proton Pump Inhbitor Formulation

1. Load the drug loaded instant release mutiparticulates in to a suitable fluidized coating machine (e.g Glatt).

2. Spray the Polymer coating solution on to the drug loaded instant release multiparticulates.

3. Once the required amount of polymer coating solution has been applied, dry the product in the fluidized coating machine.

4. Encapsulate the product in a hard fluidizede capsule using an automated encapsulation machine, sufficient to obtain 20 mg dose of Omeprazole per capsule.

(D) Delayed Release/Modified Release Prton Pump Inhibitor Dosage Form

Alternatively, once the proton pump modified release dosage form has been achieved this dosage form can be further coated with one of the polymer systems described below to produce a delayed release followed by a modified release of the proton pump inhibitor.

A non-limiting example is shown in Table 16, below, and the following, non-limiting, exemplary procedure which follows it.

TABLE 16

| | Batch | Batch | Batch | Batch | Batch |
|---|---|---|---|---|---|
| Ingredient | (g) | (g) | (g) | (g) | (g) |
| Omeprazole | 1000 | 1000 | 1000 | 1000 | 1000 |
| modified | | | | | |
| release | | | | | |
| multiparticulates | | | | | |
| *Polymer | 50 | 100 | 200 | 300 | 400 |
| Solution | | | | | |
| (A, B, C or D) | | | | | |
| TOTAL | 1050 | 1100 | 1200 | 1300 | 1400 |
| *This represents the amount of solid content in the polymer solution as the solvent is removed during processing. The amount of solids applied can be adjusted depending on | | | | | |
| # the type of dissolution profile that is required. Increased amounts of polymer solids will produce decreasing dissolution profiles. | | | | | |

Manufacturing Process—Delayed/Modified Release Omeprazole Formulation

1. Load the Omeprazole modified release mutiparticulates in to a suitable fluidized coating machine (e.g Glatt).

2. Spray the Polymer coating solution on to the Omeprazole modified release multiparticulates.

3. Once the required amount of polymer coating solution has been applied, dry the product in the fluidized coating machine.

4. Encapsulate the product in a hard fluidizede capsule using an automated encapsulation machine, sufficient to obtain 20 mg dose of Omeprazole per capsule.

While the invention has been described in connection with certain preferred embodiments so that aspects thereof may be more fully understood and appreciated, it is not intended to limit the invention to these particular embodiments. On the contrary, it is intended to cover all alternatives, modifications and equivalents as may be included within the scope of the invention as defined by the appended claims.

1. A pharmaceutical formulation comprising at least one proton pump inhibitor structured and arranged to provide an initial pH independent time-based delayed-release, and a subsequent extended-release of the at least one proton pump inhibitor, said initial pH independent time-based delayed-release period of the at least one proton pump inhibitor comprising release of at most about 20% of the at least one proton pump inhibitor during a period of about 1 to 4 hours, and the subsequent extended-release of the proton pump inhibitor being over a period of about 3 to 12 hours, and providing an hourly increase in percent release of the at least one proton pump inhibitor during any and all one hour periods of time of less than about 35%.

2. The pharmaceutical formulation according to claim 1, wherein said initial pH independent time-based delayed-release of the at least one proton pump inhibitor comprises release of less than about 10% of the at least one proton pump inhibitor during about 2 to 3 hours, and the subsequent extended-release of the proton pump inhibitor providing an hourly increase in percent release of the at least one proton pump inhibitor of less than about 30%.

3. The pharmaceutical formulation according to claim 1, wherein said initial pH independent time-based delayed-release of the at least one proton pump inhibitor comprises release of less than about 10% of the at least one proton pump inhibitor during about 2 to 3 hours, and the subsequent extended-release of the proton pump inhibitor providing an hourly increase in percent release of the at least one proton pump inhibitor of less than about 25%.

4. The pharmaceutical formulation according to claim 1, wherein said initial pH independent time-based delayed-release of the at least one proton pump inhibitor comprises release of less than about 10% of the at least one proton pump inhibitor during about 2 to 3 hours, and the subsequent extended-release of the proton pump inhibitor providing an hourly increase in percent release of the at least one proton pump inhibitor of less than about 20%.

5. The pharmaceutical formulation according to claim 1 wherein the initial pH independent time-based delayed-release of the at least one proton pump inhibitor comprises no release of the at least one proton pump inhibitor.

6. The pharmaceutical formulation according to claim 1 wherein the initial pH independent time-based delayed-release of the at least one proton pump inhibitor comprises release of less than about 5% of the least one proton pump inhibitor.

7. The pharmaceutical formulation according to claim 1 wherein the initial pH independent time-based delayed-release of the at least one proton pump inhibitor comprises release of less than about 5% or no release of the at least one proton pump inhibitor for at least about 1 hour.

8. The pharmaceutical formulation according to claim 1 wherein the initial pH independent time-based delayed-release of the at least one proton pump inhibitor comprises release of less than about 5% or no release of the at least one proton pump inhibitor for at least about 2 hours.

9. The pharmaceutical formulation according to claim 1 wherein the initial pH independent time-based delayed-release of the at least one proton pump inhibitor comprises release of less than about 5% or no release of the at least one proton pump inhibitor for about 2 to 4 hours after administration to a mammal.

10. The pharmaceutical formulation according to claim 1 wherein the subsequent extended-release of the at least one proton pump inhibitor comprises release of the at least one proton pump inhibitor over a period of from about 3 to 9 hours.

11. The pharmaceutical formulation according to claim 1 wherein the subsequent extended-release of the at least one proton pump inhibitor comprises release of the at least one proton pump inhibitor over a period of from about 4 to 9 hours.

12. The pharmaceutical formulation according to claim 1 wherein the subsequent extended-release of the at least one proton pump inhibitor comprises release of the at least one proton pump inhibitor over a period of from about 4 to 6 hours.

13. The pharmaceutical formulation according to claim 3 wherein the subsequent extended-release of the at least one proton pump inhibitor comprises release of the at least one proton pump inhibitor over a period of from about 4 to 6 hours.

14. The pharmaceutical formulation according to claim 1 wherein the formulation comprises a diffusion control system.

15. The pharmaceutical formulation according to claim 14 wherein the diffusion control system comprises at least one core including the at least one proton pump inhibitor, a diffusion control coating and a pH independent delayed-release coating.

16. The pharmaceutical formulation according to claim 15 wherein the at least one core comprises a plurality of cores.

17. The pharmaceutical formulation according to claim 16 wherein the cores are in the form of microparticles.

18. The pharmaceutical formulation according to claim 13 wherein the formulation comprises a diffusion control system, and the diffusion control system comprises a core including the at least one proton pump inhibitor, a diffusion control coating and a pH independent delayed-release coating.

19. The pharmaceutical formulation according to claim 1 wherein the formulation comprises a matrix system.

20. The pharmaceutical formulation according to claim 13 wherein the formulation comprises a matrix system.

21. The pharmaceutical formulation according to claim 1 wherein the formulation comprises an osmotic system.

22. The pharmaceutical formulation according to claim 1 wherein the formulation includes an insoluble polymer.

23. The pharmaceutical formulation according to claim 1 wherein the formulation does not include an enteric coating.

24. The pharmaceutical formulation according to claim 1 wherein the formulation does not include a disintegrant.

25. A proton pump inhibitor formulation having a dissolution profile, using a rotating paddle apparatus (USP II) using 900 ml of USP phosphate buffer (pH 6.8) at 37° C. and an agitation speed of 50 rpm of:
2 hours—≦30%,
3 hours—≦60%,
6 hours—≧20%,
8 hours—≧40%, and
12 hours—≧70%.

26. The proton pump inhibitor formulation according to claim 25 wherein the formulation is a pH independent time-based delayed-release, extended-release formulation.

27. The proton pump inhibitor formulation according to claim 26 wherein the dissolution profile is:
2 hours—less than 20%,
3 hours—greater than 10% but less than 30%,
4 hours—greater than 20% but less than 40%,
6 hours—greater than 40% but less than 60%,
8 hours—greater than 60% but less than 80%, and
12 hours—greater than 80%.

28. The proton pump inhibitor formulation according to claim 26 wherein the dissolution profile is:
3 hours—<20%,
4 hours—greater than 10% but less than 30%,
6 hours—greater than 30% but less than 50%,
8 hours—greater than 50% but less than 70%, and
12 hours—greater than 75%.

29. The proton pump inhibitor formulation according to claim 26 wherein the dissolution profile is:
4 hours—<20%,
6 hours—greater than 20% but less than 40%,
8 hours—greater than 40% but less than 60%, and
12 hours—greater than 70%.

30. A method of treating nocturnal acid breakthrough comprising orally administering a pharmaceutical formulation to a mammal, wherein said pharmaceutical formulation comprises at least one proton pump inhibitor structured and arranged to provide an initial pH independent time-based delayed-release, and a subsequent extended-release of the at least one proton pump inhibitor.

31. The method according to claim 30 wherein the mammal is a human.

32. A method of treating nocturnal acid breakthrough comprising orally administering the formulation recited in claim 1 to a human.

33. A method of treating nocturnal acid breakthrough comprising orally administering the formulation recited in claim 3 to a human.

34. A method of treating nocturnal acid breakthrough comprising orally administering the formulation recited in claim 15 to a human.

35. A method of producing a formulation including at least one proton pump inhibitor, comprising including at least one proton pump inhibitor with pharmaceutical ingredients to provide an initial pH independent time-based delayed-release, and a subsequent extended-release formulation according to claim 1.

36. A method of producing a formulation including at least one proton pump inhibitor, comprising including at least one proton pump inhibitor with pharmaceutical ingredients to provide an initial pH independent time-based delayed-release, and a subsequent extended-release formulation according to claim 3.

37. A method of producing a formulation including at least one proton pump inhibitor, comprising including at least one proton pump inhibitor with pharmaceutical ingredients to provide an initial pH independent time-based delayed-release, and a subsequent extended-release formulation according to claim 15.

38. A formulation including at least one proton pump inhibitor, said formulation having a Tmax of greater than 3.5 hours.

39. The formulation according to claim 38 wherein Tmax is greater than about 4 hours.

40. The formulation according to claim 38 wherein Tmax is about 4 to 12 hours.

41. The formulation according to claim 38 wherein the formulation is a pH independent time-based delayed-release, extended-release formulation.
